(19) United States
(12) Patent Application Publication
(10) Pub. No.: US 2007/0292844 A1
(43) Pub. Date: Dec. 20, 2007

(54) Enhanced biohazard detection system

(75) Inventors: David J. Tilles, Woodstock, MD (US); Matthew W. Snyder, Baltimore, MD (US); Kenneth S. Damer, Parkville, MD (US); Alfred R. Monch, Columbia, MD (US); Karen G. Jarvis, Rock Stream, NY (US)

Correspondence Address: ROTHWELL, FIGG, ERNST & MANBECK, P.C., 1425 K STREET, N.W., SUITE 800, WASHINGTON, DC, 20005, US

(73) Assignee: Northrop Grumman Corporation, Los Angeles, CA (US)

(21) Appl. No.: 11/723,865

(22) Filed: Mar. 22, 2007

Related U.S. Application Data
(60) Provisional application No. 60/784,453, filed on Mar. 22, 2006.

(51) Int. Cl. C12Q 1/70; C12M 1/36; F15B 15/00; G01N 35/02; G01N 33/53; C12Q 1/00

(52) U.S. Cl. 435/5; 134/195; 134/61; 422/63; 435/286.5; 435/4; 435/7.1; 436/50

(57) ABSTRACT

An enhanced biohazard detection system and a method for detecting, screening and analyzing biological agents are disclosed. The system incorporates an immunoassay detection device to provide a more robust, less expensive method for detecting biological agents in air samples collected at strategically located monitored sites. The immunodetection based assays may be used either in parallel with, or as a prescreening assay for, a polymerase chain reaction based assay for detecting and identifying biological agents in air samples.

### CROSS-REFERENCE TO RELATED APPLICATIONS

This application claims priority to U.S. Provisional Patent Application Ser. No. 60/784,453, filed on Mar. 22, 2006, the contents of which are incorporated herein in their entirety.

### BACKGROUND OF THE INVENTION

1. Field of the Invention

The present invention relates to an enhanced biohazard detection system for detecting, screening and analyzing biological agents such as bacteria, viruses and toxins.

2. Discussion of the Background Art

Biohazard detection systems for detecting, screening and analyzing biological agents such as anthrax have been described. For example, U.S. Patent Publication No. 2004/0063198, the disclosure of which is incorporated herein by reference in its entirety, describes a point source biological agent detection system designed to detect aerosolized biological agents from an air sample. The system of Publication No. 2004/0063198 uses polymerase chain reaction technology (PCR) to detect bacterial agents such as Bacillus anthracis, the agent which causes anthrax, in air samples collected at strategic collection points. In one application air samples are collected around high speed equipment, such as the high speed mail sorting equipment used by the United States Postal Service to detect anthrax that may be present in mail and released into the air as the mail moves through processing equipment.

As presently configured, the prior art system comprises a PCR bio-identifier system that includes automated sample and detection processing. The PCR system consists of two components, a PCR analysis instrument and a disposable multi-chamber cartridge that is inserted into the instrument. The critical reagents required by the PCR instrument are loaded into the cartridges at the factory to avoid handling of sensitive reagents by an operator and must be replaced at frequent intervals to assure that adequate supplies of reagents are available for assays.

The cost of consumables used in the PCR analysis makes the prior art system expensive to operate. In addition, the configuration of the PCR analysis instrument does not allow the system to accommodate detection assays that may be required to detect agents such as toxins that are not detectable by PCR. PCR based assays are limited in that they can only detect nucleic acid based samples from particular bacteria and viruses. Currently, the system detects only anthrax.

Thus, there continues to be a need for a more efficient, less costly biohazard detection system that can provide enhanced performance and rapidly detect a larger number of biohazards, including bacteria, viruses and toxins.

### SUMMARY OF THE INVENTION

A preferred embodiment of the present invention provides a biohazard detection system that utilizes an immunoassay device to detect bacteria, viruses and toxins. This embodiment enhances the capabilities of the prior art PCR based systems by providing an efficient prescreen for the presence of a biohazard in a sample before it is subjected to PCR analysis. The system further provides orthogonal analysis of biological agents, such as bacteria and viruses, and detection of toxins, reduces operating costs and provides enhanced sample processing capabilities.

In a first aspect an embodiment of the present invention provides an enhanced biohazard detection system with a first sample analysis device that is an immunoassay based biological sample analyzer or device and a second sample analyzer that is a PCR based biological sample analyzer. The immunoassay device analyzes particles in collected air samples using immunoassay detection technology.

In a second aspect an embodiment of present invention provides an enhanced biohazard detection system that allows detection of toxins, as well as bacteria and viruses. The immunoassay may be carried out using an immunoassay device that comprises one or more antibodies directed to antigens of a bacteria, virus or toxin.

In a third aspect an embodiment of the present invention may provide an orthogonal or two-dimensional detection system, where a first analysis step detects the presence of a biohazard in a prescreen assay using immunodetection assay technology, and a second analysis step identifies the particular biohazard agent detected in the prescreen. In one preferred embodiment a sample is tested using the assay of the PCR detection step only after a preliminary positive signal is obtained from the first immunoassay device. The use of PCR assays to confirm the presence of a biohazard agent provides significant cost savings for routine monitoring with a biohazard detection system. In addition the use of an orthogonal or two-dimensional detection system will improve the accuracy of monitoring, resulting in fewer false positive results.

In a fourth aspect an embodiment of the present invention provides a system for testing a bacteria, virus or toxin biohazard by an immunoassay, particularly a biohazard agent for which no PCR based assay exists in which the immunoassay device may be configured to perform a second analysis, which may include a different immunoassay, to provide confirmation of a first preliminary positive identification.

In a fifth aspect an embodiment of the present invention provides an enhanced fluidics handling module (EFHM) which may be incorporated into the biohazard detection system to allow a sample to be tested in an immunoassay device and in a PCR assay device, in parallel or in series. The enhanced fluidics module provides for testing and retesting of two or more samples pooled and stored in a second reservoir.

In a sixth aspect the present invention provides an enhanced mission configurable fluidics module (MCFM) that allows for variable configuration of the biohazard detection system with minimal or no changes to the physical components of the system. This configurable system may be readily modified to incorporate new assay technologies, for example, technologies for detecting a biological agent. The module may also be easily configured to accommodate the use of additional reagents during sample collection and analysis. For example, a surfactant may be added to a sample to prevent the binding of protein toxins to material in a sample fluid or to component surfaces with which the sample may come in contact.

In a seventh aspect the present application provides a method for detecting agents using an immunoassay detection system comprising collecting an aerosolized sample at a strategically selected point location or locations; producing a liquid sample from the aerosolized air sample; transporting and delivering a portion or aliquot of the liquid sample to a sample reservoir in a configurable fluidics module, where the sample is stored prior to being moved to either an immunoassay detection device or a PCR detection device, or both. In one preferred embodiment the fluidics module may further contain a second sample reservoir in which a sample may be combined with one or more other samples and stored prior to being testing a second time to confirm a first test result. In one preferred embodiment the biohazard detection system may comprise a mission configurable fluidics module 300, with a configuration which can be adapted to accommodate different applications with minimal or no changes to the physical system.

The above features and advantages of the present invention, as well as the structure and operation of preferred embodiments, are described in more detail below with reference to the accompanying drawings.

### BRIEF DESCRIPTION OF THE DRAWINGS

The accompanying drawings, which are incorporated herein and form part of the specification, help illustrate various embodiments of the present invention and, together with the description, further serve to explain the principles of the invention and to enable a person skilled in the pertinent art to make and use embodiments of the invention. In the drawings, like reference numbers indicate identical or functionally similar elements.

FIG. 1 is a schematic diagram of an enhanced biological detection system according to an embodiment of the present invention.

FIG. 2 is a schematic diagram of an enhanced fluidics handling module for use in an enhanced biological detection system according to the present invention.

FIG. 3 is a perspective view of a mission configurable fluidics module for use in an enhanced biological detection system according to the present invention.

### DETAILED DESCRIPTION OF PREFERRED EMBODIMENTS

In one embodiment the subject matter of the present invention provides an enhanced biohazard detection system that incorporates a device for detecting biological agents using immunoassay detection technology. The immunodetection assay may be used as a prescreen assay to make the present PCR-based systems more efficient and economical to operate or as a stand alone assay to detect the presence of biohazardous agents that cannot be detected using the PCR detection assays. In addition, it may be used to detect the presence of biohazardous agent in parallel to the PCR detection assays as a method of improving accuracy and reducing the system false alarm rate.

In a further embodiment the present invention provides an enhanced biohazard detection system having an enhanced fluidics module that allows a collected sample to be tested with a PCR device and/or an immunoassay device, in parallel, in series, or after two or more samples are pooled. In one embodiment the system is used to test air surrounding moving equipment that transports items that may be contaminated with biohazardous agents, such as pieces of mail.

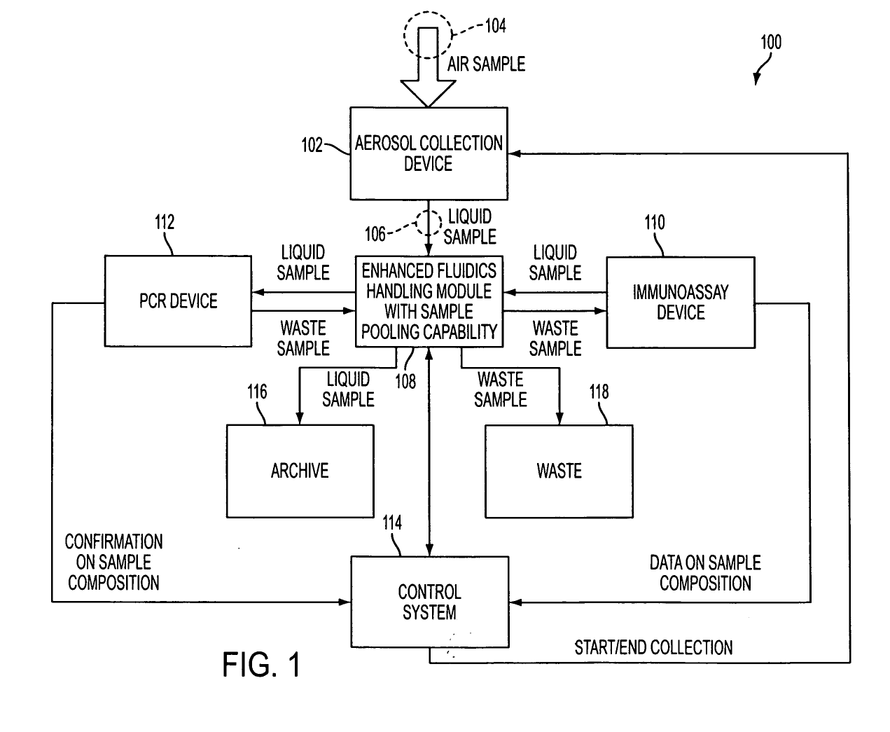

A schematic diagram of an enhanced biohazard detection system 100 according to the present invention is shown in FIG. 1. The system 100 includes a collection device 102 that draws an air sample 104 from an area to be sampled and a fluidics handling module 108 that provides a means for a collected sample to be tested in an immunoassay detection device 110 and/or a PCR detection device 112. System 100 also includes a control system 114, a waste storage area 118 and an optional archive storage area 116. In one embodiment, the air sample 104 is drawn through an anti-static, smooth bore flexible collection hose into a suitable filter, such as, for example, a dry cyclone filter. In a preferred embodiment, the sample is drawn through a collection hood with or without a filter.

In one embodiment of the aerosol collection device 102, particles in the air sample 104 are collected in a liquid to produce a liquid sample 106 for analysis. A suitable collection device is described in U.S. Patent Publication No. 2004/0063198, although an appropriate collector-concentrator that is efficient for collecting bio-aerosols, particulate matter and soluble vapors can be used. In one embodiment, the collection device 102 may be a SpinCon® collection and concentration system developed by Sceptor Industries to collect bio-aerosols, particulate matter and soluble vapors at strategically placed locations. Alternatively, separate collection and concentration devices can be used.

The collection device 102 can advantageously be placed at a location adjacent to a transport path for items that may be contaminated with a biological agent, for example, adjacent mail sorting and handling equipment.

In a preferred embodiment of the collection process, a collection fluid is injected into a vertical glass tube or contactor of the aerosol collector-concentrator. Air is drawn into the contactor through a slit partially covered by a collection fluid. The fluid across the slit is atomized into small water droplets by the incoming air, greatly increasing the surface area in contact with the air. In one embodiment the collector-concentrator impinges the air into about 12 milliliters (ml) of a collection fluid, which can be a variety of different fluid types. In one preferred embodiment the collection fluid may be de-ionized sterile water, which has been filtered through a 0.2 μm filter. After particles in the air sample are picked up by the liquid, the sample, which is now a liquid sample, is transferred from the collector to a fluidics module 108.

The fluidics handling module 108 provides means for a collected sample to be tested in an immunoassay detection device 110 and/or a PCR detection device 112 by storing and delivering aliquots of the liquid sample 106 to receptacles in which detection and identification assays are carried out. The fluidics handling module 108 allows the collected sample to be tested in both modules in parallel, or to be tested in first one module, then the second module, in series.

The system immunoassay detection device 110 detects the reaction products of an immunodetection assay on a first portion or aliquot of the original sample 106 from a first or second reservoir in the fluidics handling module 108. The immunoassay detection device 110 may have one, two or an array of photodiode detectors, or another means of measuring light, such as a CCD camera. The device may also include an automated plate carriage for transporting a multi-well plate from a liquid fill area to a position under the light measuring component(s). The device may also include an interface for reporting the test results provided by the immunoassay device to the control system 114 of the biohazard detection system.

In preferred embodiments, the immunoassay device 110 provides an antibody based assay in which antibodies that recognize and capture antigens present on biohazardous agents are used to detect the presence of the agents in samples of particles taken from air. The immunodetection assay utilizes antibodies directed to known biohazardous agents, such as agents designated as Bioterrorism Agents by governmental agencies such as the Center for Disease Control. Such biohazardous agents may include, for example, bacteria that cause anthrax (Bacillus anthracis), botulism (Clostridium botulinum), tularemia (Francisella tularensis) or plague (Yersinia pestis). The assay may also use antibodies directed at viruses, such as smallpox virus (variola major) and filoviruses and arenaviruses that cause viral hemorrhagic fevers. In addition, the system may use antibodies directed at toxins such as the Epsilon toxin of Clostridium perfringens and the Ricin toxin from Ricinus communis.

In preferred embodiments, the assay of the immunoassay device 110 may be any immunoassay method in which the final read-out detects an antibody-antigen reaction by measuring a signal produced using a reagents and appropriate labels where detection of the label can be correlated with the presence of an antigen of interest. Suitable detection methods include colorimetry, fluorescence, radioactivity and chemiluminescence. Preferred methods include fluorescence and chemiluminescence. In some preferred embodiments electrochemiluminescence detection methods may be used. Immunoassay devices known to those of skill in the art may be adapted for use in the enhanced biological detection system of the present invention. For example, the Sector PR devices of Meso Scale Discovery or the Luminex System of Luminex Corporation may be adapted for use as a subsystem in the enhanced biological detection system 100.

Immunoassays such as enzyme linked immunosorbent assays (ELISAs) that are known in the art may be readily adapted for use in the immunoassay device. Enzyme conjugated antibodies may be used in automated assays include horseradish peroxidase and alkaline phosphatase. Useful substrates for detecting bound antibodies include colorimetric, fluorometric, and chemiluminescent substrates that provide very high sensitivity and low background signals and allow accurate detection of a specific bacteria, virus or toxins, which may be present in very low amounts in a sample.

The immunoassay device may include an automated filling station at which samples and other reagents are added to an assay reservoir, such as the well of a microtiter plate. In one preferred embodiment the immunoassay reservoir is one or more wells of a standard ninety-six well microtiter plate. The filling station may further include a probe or pipette that is connected to the fluidics handling module 108 which delivers a liquid sample 106 to the assay reservoir or well. The fluidics handling module may also store and deliver enzyme linked secondary antibodies and reagents for developing the immunodetection assay. A photometer or other detection device is used for measuring emitted light, fluorescence or chemiluminescence produced by enzymes linked antibodies such as those known to those of skill in the antibody art when those antibodies are bound to the agent of interest. A carriage mechanism may be used to move an assay plate from a liquid filling station to the photometer or detection device.

In one embodiment the biohazard detection system provides a method of detecting a biohazard using an immunoassay as a prescreen assay. When the immunoassay device 110 detects the presence of a bacterial or viral biohazard, the apparatus sends a “preliminary positive” signal to the control system 114, which may include one or more computers. The control system 114 may then send a signal to the fluidics module 108 to transfer a second portion or aliquot of the original sample from the first sample reservoir, to a second analysis apparatus 112 which may be a PCR-based biological agent identifier system. One PCR method that may be used is described in detail in U.S. Patent Publication No. 2004/0063198. One of skill in this art may identify or design other suitable PCR assay devices (or devices that incorporate other detection technologies with similar or better sensitivity and specificity as PCR) for incorporation into the biohazard detection system.

In one embodiment, the PCR-based biological agent identifier system 112 consists of two components, a multi-chamber cartridge and a PCR analysis instrument. On receipt of a signal from the control system 114, the fluidics handling module 108 transfers a portion or aliquot of the original sample from its reservoir into the multi-chamber cartridge for confirmation of the preliminary positive signal by a PCR assay. In one preferred embodiment the PCR assay device extracts nucleic acid from material present in the sample prior to analyzing extracted nucleic acids by PCR using methods known in the art. The PCR detection apparatus may be set to run a series of tests using different sets of DNA probes and primer pairs designed for individual biohazard agents to confirm the presence of and identity of the agent in the sample.

If the immunodetection assay identifies a bacteria, virus or toxin biohazard for which no PCR test exists, a second immunoassay analysis may be performed, preferably using a different antibody or set of antibodies to confirm or verify the preliminary positive result of the first immunoassay.

In one preferred embodiment the biological detection system 100 of the present invention advantageously provides a method for identifying a toxin in the sample of particles collected from a monitored location. Because toxins are generally not nucleic acids, the presence of a toxin cannot be confirmed with a nucleic acid based PCR detection assays. PCR assays can detect only the presence of a bacteria or other organism that produces a toxin. Thus, in one embodiment, an immunoassay device 110 will be used to confirm the presence of a toxin by performing a second immunodetection assay using an antibody that may differ from the antibody of the first screening assay.

An unused liquid sample 106 may be held in the sample reservoir of the fluidics handling module 108 until analysis is complete. When a particular sample 106 is determined to be positive for a biohazard, the remaining sample may be transferred to an archive storage area or container 116, as shown in FIG. 1, and eventually be removed from the biohazard detection system 100 for further testing. Liquids from analysis assays may be transferred to a waste storage area or container 118 as shown in FIG. 1.

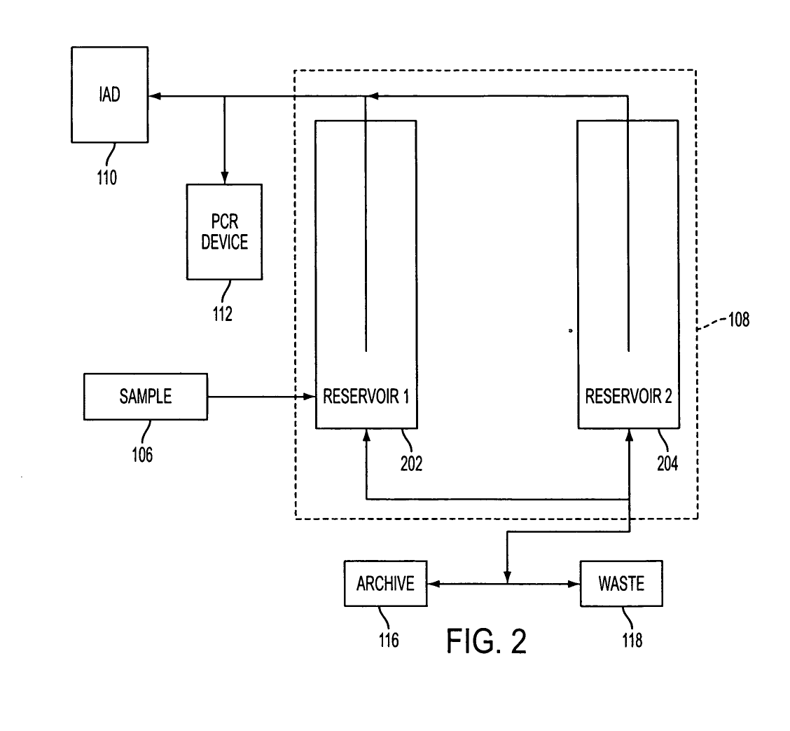

In one application two or more samples may be pooled and held for further testing to confirm the results of a first detection test by use of a fluidics handling module with a plurality of reservoirs. In one embodiment, shown in FIG. 2, the fluidics handling module 108 includes a first reservoir 202 and a second reservoir 204. A first portion or aliquot of the sample from reservoir 202 is tested using the immunoassay device 110 as described above. Samples which test negative for a biohazard agent in the immunodetection assay can be collected in the second sample reservoir 204, which may serve as a pooling reservoir. Pooled samples may be stored and subsequently tested using the PCR detection device to confirm the results of a prescreen immunodetection assay. Alternatively, the pooled samples may be tested in a second immunodetection assay.

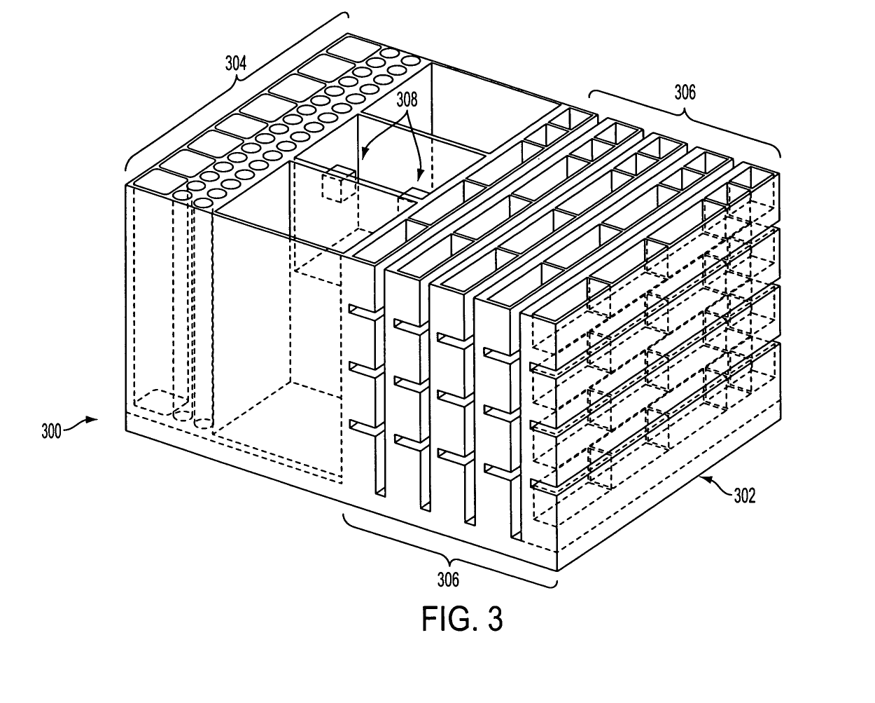

In one embodiment the enhanced fluidics module 108 is a mission configurable fluidics module 300 such as the module 300 of FIG. 3. The configurable layout of module 300 allows the biohazard detection system to be readily reconfigured should it be desirable to use the system to detect a new agent, to incorporate a new detection assay or to otherwise modify the assays carried out by the system.

In a preferred embodiment, the configurable fluidics module 300 includes a manifold 302 that forms a common base which can be customized to have reservoirs for the specific solutions required by a detection system. The manifold may contain large volume reservoirs 304 that can be used to store consumables, such as, water, test buffers and reagents which are used in large volumes relative to standard sample volumes.

Small volume fluid reservoirs 306 may be used for holding and storing samples. The small volume fluid reservoirs 306 may also be used for storing consumables, particularly in multiple, smaller volumes. Smaller volume reservoirs may be equipped with volume sensors and multiple extraction points to allow for the processing of samples from different points within a water column using methods known to those of skill in the art. Consumables and samples can be transported to and from reservoirs 304 and 306 using fluid handling methods and mechanisms known to those of skill in the art. For example, the configurable fluidics module 300 can include one or more onboard pumps 308 in fluid communication with the reservoirs, e.g., via conduits integrally formed in the manifold and/or other parts of the module such that the reservoirs are operably connected to one another. Various valves and sensors can also be provided as part of the fluidics module 300 to control the flow of fluids to and from the reservoirs as well as to provide information to the system about reservoir conditions (e.g., fluid level, temperature, etc.) and the fluids contained therein. Alternatively, the fluid handling mechanism may include an automated pipette device movable between reservoirs and/or detection devices. In one embodiment, the reservoirs for smaller volumes of solution may be modular (i.e., line replaceable units) so that they are exchangeable with other reservoir modules. Exchangeable modules allow for easy cleaning or replacement if changes to the system exceed the inherent variability of the system.

A complementary fluidics loading station (not shown) can be provided for a quick changeout of all consumables to facilitate loading. Optionally, the loading station may be connected to the manifold system.

The software used to program the computer control system 114 of the biohazard detection system may include an address for each reservoir and to allow easy modification of fluid routing from application to application or detection assay to detection assay.

The mission configurable fluidics module 306 may further comprise a long, thin pump cylinder that allows for accurate measurements and long service life. The piston of the pump may serve as a neutral reservoir for transfers of fluids or solutions. In one embodiment metering of volumes occurs only on injection, and not on aspiration of fluid. In this embodiment more fluid will be drawn into the pump cylinder than the small volume that will be distributed or injected. Thus, the remainder of any uninjected fluid will be returned to a starting reservoir or sent to a waste reservoir.

To mitigate failures, one embodiment of the mission configurable fluidics module may contain redundant pumps 308.

In one embodiment the configurable fluidics module 108 may contain a cleaning or rinsing reservoir of gray, i.e., recycled, water that will allow for multiple rinses of the system without consuming large quantities of water. Gray water will be sent to the waste reservoir and replaced after a determined number of rinses are done.

The enhancements provided by the mission configurable fluidics module allows for more economical use of consumables and a lower overall cost of operation for the biohazard detection system. The variable configuration allows the detection system to be easily adapted to different detection assays with minimal or no changes to the system. For example, the module can be adapted to provide a surfactant that will prevent binding of toxins or proteins to material present in a liquid sample. The surfactant may be stored in a large volume reservoir 304 of the module and injected directly into the aerosol collection device during sample collection in controlled metered doses.

While various embodiments/variations of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalents.

What is claimed is:

1. An automated biological agent detection system comprising:
an aerosol collection device positionable at one or more point locations to collect an aerosol sample;
a concentrator in communication with said aerosol collection device to receive the aerosol sample and produce a liquid sample from the aerosol sample;
an immunoassay detection device;
a fluidics handling module including one or more reservoirs for storing the liquid sample and a fluid handling mechanism; and
a control apparatus providing automated control of the fluid handling mechanism so that a first portion of the liquid sample is transferred from said fluidics handling module to said immunoassay detection device.

2. The system of claim 1, further comprising a second detection device.

3. The system of claim 1, wherein said immunoassay detection device provides a prescreen assay for detecting the presence of a biological agent.

4. The system of claim 2, wherein said control apparatus sends a signal to said fluidics handling module to transfer a second portion of a sample to said second detection device in response to a positive signal from a prescreen immunoassay performed by said immunoassay detection device.

5. The system of claim 4, wherein said second detection device is a polymerase chain reaction based biological detection device.

6. The system of claim 1, wherein said fluidics handling module further comprises a fluidics loading station for water, buffers and reagents for use in a detection assay.

7. The system of claim 1, wherein said fluidics handling module further comprises a customizable design that can be adjusted to handle multiple volumes of reagents for sample processing and assay.

8. The system of claim 1, wherein the biological agent is a bacterium.

9. The system of claim 1, wherein the biological agent is a virus.

10. The system of claim 1, wherein the biological agent is a toxin.

11. A method of detecting a biological agent comprising:
collecting an aerosol sample of air at one or more locations;
producing a liquid sample from the aerosol;
delivering a portion of the liquid sample to one or more reservoirs of an assay device;
analyzing the sample in an immunoassay detection device to detect the presence of a biological agent; and
sending a signal to a biological detection system control system having overall automated control of the method.

12. The method of claim 11, wherein the step of detecting the presence of a biological agent is a prescreening step.

13. The method of claim 11, wherein the liquid sample is exposed to an antibody that binds a bacterial antigen.

14. The method of claim 13, wherein the liquid sample is exposed to antibody that identifies bacteria that cause anthrax.

15. The method of claim 13, wherein a particle liquid sample is exposed to antibody that identifies bacteria that cause plague.

16. The method of claim 13, wherein a particle liquid sample is exposed to antibody that identifies bacteria that causes tularemia.

17. The method of claim 11, wherein particles in the liquid sample are exposed to an antibody that binds a virus.

18. The method of claim 18, wherein the antibody identifies the smallpox virus.

19. The method of claim 11, wherein particles in the liquid sample are exposed to an antibody that binds a toxin.

20. The method of claim 19, wherein a particle liquid sample is exposed to antibody that identifies toxin that cause botulism.

21. The method of claim 19, wherein the toxin is ricin.

22. The method of claim 11, wherein a control device responds to a positive signal from an immunoassay by signaling the fluidics module to transfer a second portion of the prescreened liquid sample to the receptacle of a biological agent identifier where the prescreened positive sample is further analyzed to identify the biological agent.

23. The method of claim 22, wherein a prescreened positive sample is analyzed in a PCR based assay.

24. The method of claim 22, wherein a prescreened positive sample is analyzed in a second immunodetection assay.

25. The method of claim 11 further comprising analyzing the sample in parallel in PCR detection device.

26. A method of decreasing the operating costs of a biological detection system for analyzing a sample of particles in air comprising prescreening the sample for the presence of biological agents in an immunodetection assay.

27. A method of extending the maintenance interval for replacing consumables in a biological detection system for biohazardous agents comprising prescreening the sample for the presence of biological agents in an immunodetection assay.

28. A configurable fluidics module comprising a manifold providing a common base for multiple reservoirs of varying size that may be operably connected by a pump such that it is possible to pump a fluid from any reservoir to any other reservoir.

29. The fluidics module of claim 28, further comprising a first sample reservoir for a liquid sample from an aerosol collection device.

30. The fluidics module of claim 29, further comprising at least one reservoir for storing a sample determined to be positive in a first immunodetection assay.

31. The fluidics module of claim 28, wherein the reservoirs are capable of volume sensing and extraction based on the volume of liquid in the reservoir.

32. The fluidics module of claim 31, wherein the volume of liquid in a reservoir is monitored by a control system.

33. The fluidics module of claim 28, wherein the pump comprises a thin pump cylinder with a piston that is a neutral reservoir in transfers.

34. The fluidics module of claim 33, wherein the pump meters liquid on injection.

35. The fluidics module of claim 28 further comprising a reservoir of rinse water for multiple rinses of components of a biological detection system, wherein the rinse water is replaced after a predetermined number of rinses.
